(19) United States
(12) Patent Application Publication
(10) Pub. No.: US 2022/0183754 A1
(43) Pub. Date: Jun. 16, 2022

(54) SURGICAL DEVICE, LASER SURGERY SYSTEM, AND METHODS OF OPERATION THEREOF

(71) Applicant: LightScapel LLC, Bothell, WA (US)

(72) Inventors: Peter VITRUK, Bothell, WA (US); Soroush ZAGHI, Bothell, WA (US)

(21) Appl. No.: 17/441,651

(22) PCT Filed: Mar. 20, 2020
(86) PCT No.: PCT/US2020/023969

§ 371 Date: Sep. 21, 2021

Related U.S. Application Data
(60) Provisional application No. 62/826,793, filed on Mar. 29, 2019.
(60) Provisional application No. 62/822,726, filed on Mar. 22, 2019.

(51) Int. Cl. A61B 18/22; A61B 18/20

(52) U.S. Cl. CPC A61B 18/22; A61B 2018/00601; A61B 2018/2272; A61B 18/201

(57) ABSTRACT

Surgical devices and methods of operation thereof are provided herein. The surgical devices include interior walls defining a cavity that extends along a first axis and having an aperture at a first end of the surgical devices. The surgical devices include a backstop spaced apart from the aperture and having a surface opposing the aperture. The surgical devices include an arm that extends from the surgical device. The surgical devices are configured to receive a fiber waveguide into the cavity at a second end. The surgical devices of some embodiments include a surface for reflecting laser light from the fiber waveguide.

### BACKGROUND

### Technical Field

The present disclosure relates to medical devices and, more particularly, relates to surgical laser devices for incising or excising bodily tissue.

### Description of the Related Art

The use of lasers in surgical and medical settings has rapidly increased in recent decades due to the advantages of lasers over metal instruments, such as reduction in scar tissue and pain, and improvement in sanitary conditions. The design and use of devices for applying lasers to tissue in certain areas of the body has presented a complex and challenging issue.

### BRIEF SUMMARY

Embodiments of a surgical device may be summarized as including a body having an elongated shape with interior walls defining a cavity extending along a first axis and having a first aperture at a first end of the body, the cavity sized and shaped to receive a fiber waveguide; a backstop spaced apart from the first aperture and having a surface opposing the first aperture along the first axis at a distance of 6mm or less; and an arm extending between and connecting backstop and the body.

The surgical device may further include a nozzle provided at the first end and having a frustoconical shape, the first aperture located at an end of the nozzle for emitting laser light from the fiber waveguide. The surgical device may include a monolithic structure including at least the nozzle, the arm, and the backstop. The backstop may extend from the arm and intersect with the first axis, the backstop having a curved surface facing the first aperture. The backstop may have a body extending upwardly from the arm and an end portion having a hemispherical shape. The backstop may have a circular cross-sectional area and the surface opposing the first aperture may have a flat shape extending in a direction transverse to the first axis. The backstop may have a circular cross-sectional area and the surface opposing the first aperture may have a concave shape extending in a direction transverse to the first axis. The body, the backstop and the arm may be composed of the same material that forms a monolithic structure.

The surgical device may further include a wall extending between the first aperture and the arm.

The surgical device may further include a nozzle portion disposed at the first end and having a shape that tapers in cross-sectional area from the body to the first aperture, wherein the wall extends from the body to the first aperture. The arm may extend outwardly from the wall at a location spaced apart from the first aperture. The body may include a second aperture provided at a second end of the body opposite to the first end for receiving the fiber waveguide in the cavity. The body may include an attachment means located at the second aperture for selective attachment of the fiber waveguide to the surgical device. The surface of the backstop may be blasted with a stream of abrasive material. The surface of the backstop may be peened.

The surgical device may further include a light attached to the body for illuminating bodily tissue under surgical operation.

The surgical device may further include a camera attached to the body for recording surgical procedures.

A surgical device assembly may be summarized as including a body having an elongated shape and having first interior sidewalls extending from a first aperture at a first end of the body; a nozzle portion attached to the body at the first aperture and including a second aperture, the nozzle portion having second interior sidewalls tapering in cross-sectional area toward the second aperture, the first interior sidewalls and second interior sidewalls defining a cavity extending along a first axis; and a backstop portion having a backstop that includes a surface spaced apart from the second aperture at a distance of 6 mm or less and opposing the second aperture along the first axis. The nozzle portion may be selectively removable from the body. The backstop portion may include an arm extending from the body and connecting the backstop to the body.

The surgical device assembly may further include an arm that extends from and connects the backstop to the nozzle. The nozzle portion and the backstop portion may be composed of the same material that forms a monolithic structure. The cavity may be sized and shaped to receive a fiber waveguide

A method of performing a surgical operation using a surgical device that comprises a body having an elongated shape with interior walls defining a cavity extending along a first axis, and having a first aperture at a first end of the body, the cavity containing a fiber waveguide configured to emit laser light, a backstop spaced apart from the first aperture and having a surface opposing the first aperture along the first axis at a distance of 6 mm or less, and an arm extending between and connecting the body and the backstop, the method may be summarized as including positioning first bodily tissue between the first aperture and the backstop and at a distance of less than 6 mm away from the first aperture; and manually manipulating the surgical device to direct the laser light to incise or excise the first bodily tissue. The method may include manually manipulating the surgical device to direct the laser light to create an incision in the first bodily tissue; inserting at least the backstop into the incision; positioning second bodily tissue between the first aperture and the backstop; and manually manipulating the surgical device to direct the laser light to incise or excise the second bodily tissue.

A method of performing a surgical operation on a frenulum using a surgical device that comprises a surgical device body having an elongated shape with interior walls defining a cavity extending along a first axis, and having a first aperture at a first end of the body, the cavity containing an fiber waveguide configured to emit laser light, a backstop spaced apart from the first aperture and having a surface opposing the first aperture along the first axis at a distance of 6 mm or less, and an arm extending between and connecting the body and the backstop, the method may be summarized as including positioning a medial portion of the frenulum between the backstop and the first aperture; causing, via movement of the surgical device body, the laser light to make a first incision in the frenulum; causing, via movement of the surgical device body, the laser light to make a second incision in the frenulum along a direction transverse to the first incision; causing, via manipulation of the surgical device body, insertion of at least the backstop of the surgical device into the second incision; positioning, when the backstop of the surgical device is inserted into the second incision, bodily tissue of the frenulum between the backstop and the first aperture; and causing, via manipulation of the surgical device body, the laser light to incise or excise the bodily tissue. The method may include separating tissue of the frenulum by causing insertion of a member or the backstop into the second incision. The method may include probing tissue in the second incision to identify the bodily tissue to be incised or excised. The method may include gripping tissue of the frenulum with forceps. The bodily tissue incised or excised may be fascia muscle. The bodily tissue incised or excised may be genioglossus muscle fiber. The first incision may be a lateral incision in the medial portion of the frenulum. The second incision may be a vertical incision in the frenulum.

A system may be summarized as including a laser light source configured to generate laser light; an fiber waveguide including optical fiber optically coupled to the laser light source and including an fiber tip configured to emit the laser light; and a surgical device including a body having an elongated shape with interior walls defining a cavity extending along a first axis and having a first aperture at a first end of the body, the cavity sized and shaped to receive the fiber waveguide; a backstop spaced apart from the first aperture and having a surface opposing the first aperture along the first axis at a distance of 6 mm or less; and an arm extending between and connecting the backstop and the body. A focal distance of the laser light emitted from the fiber tip may be located between the first aperture and the surface of the backstop. The surgical device may be selectively attachable to a retaining portion of the fiber waveguide. The laser light source may be a carbon dioxide laser light source.

A surgical device may be summarized as including a main body having an elongated shape with first interior walls defining a cavity extending along a first axis and having an aperture at an end of the main body, the cavity sized and shaped to receive a fiber waveguide; an arm extending from the end; a backstop spaced apart from the aperture and having a first reflective surface; and a second reflective surface positioned along the first axis and oriented at an oblique angle with respect to the first axis and oriented at the oblique angle with respect to the backstop. The first interior walls may define a first portion of the cavity extending along the first axis, the main body may include second interior walls defining a second portion of the cavity extending along a second axis different than the first axis, the first reflective surface spaced apart from and opposing the aperture along the second axis. The backstop may extend from the arm in a direction transverse to the second axis, wherein the backstop, the arm, and the end define a recessed portion opening transversely relative to the second axis. The second reflective surface may be provided on the arm and oppose the aperture. The backstop may oppose the arm, and the first reflective surface may oppose the second reflective surface. The backstop may extend from the end and may be spaced apart from the arm, wherein the backstop, the arm, and the end define a recessed portion of the surgical device. The main body may include a body portion configured to receive the fiber waveguide and a nozzle portion including the arm, the backstop, and the second reflective surface, and the nozzle portion being selectively removable and attachable from and to the body portion.

A surgical device for use in a treatment. The treatment may be a frenectomy. The treatment may be a frenoplasty.

### BRIEF DESCRIPTION OF THE SEVERAL VIEWS OF THE DRAWINGS

FIG. 1 is a side view of a first handpiece used in surgical settings;

FIG. 2 is a side perspective view of a second handpiece used in surgical settings;

FIG. 3 is a side perspective view of a first surgical device according to one or more embodiments;

FIG. 4 is a side perspective view of a second surgical device according to one or more embodiments;

FIG. 5A is a cross-sectional view of the second surgical device according to one or more embodiments;

FIG. 5B is a longitudinal cross-sectional view taken along a length of the surgical device 400 according to one or more embodiments;

FIG. 6A is a cross-sectional view of a nozzle of the second surgical device according to one or more embodiments;

FIG. 6B is a longitudinal cross-sectional view taken along a length of the surgical device 400 according to one or more embodiments;

FIG. 7 is an overhead plan view of the second surgical device according to one or more embodiments;

FIG. 8 is a perspective view of a first backstop of the second surgical device according to one or more embodiments;

FIG. 9 is a perspective view of a second backstop of the second surgical device according to one or more embodiments;

FIG. 10A is a cross-sectional view of a third backstop of the second surgical device according to one or more embodiments;

FIG. 10B shows a longitudinal cross-sectional view of the second surgical device according to one or more embodiments;

FIG. 11A is a side view of a third surgical device according to one or more embodiments;

FIG. 11B is a cross-sectional view of the third surgical device;

FIG. 11C is a cross-sectional view of the third surgical device;

FIG. 11D is a cross-sectional view of the third surgical device;

FIG. 12A is a side view of a fourth surgical device according to one or more embodiments;

FIG. 12B is a cross-sectional view of the fourth surgical device;

FIG. 12C is a side view of the fourth surgical device;

FIG. 12D is a cross-sectional view of the fourth surgical device;

FIG. 13 is a laser surgery system according to one or more embodiments;

FIG. 14 is a first surgical operation using a surgical device according to one or more embodiments;

FIG. 15 is a second surgical operation using a surgical device according to one or more embodiments;

FIG. 16 is a third surgical operation using a surgical device according to one or more embodiments;

FIG. 17 is a fourth surgical operation using a surgical device according to one or more embodiments;

FIG. 18 is a fifth surgical operation using a surgical device according to one or more embodiments; and

FIG. 19 is a sixth surgical operation using a surgical device according to one or more embodiments.

### DETAILED DESCRIPTION

The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure have not been shown or described in order to avoid unnecessarily obscuring descriptions of the embodiments. Additionally, the various embodiments may be methods, systems, or devices.

The term “herein” refers to the specification, claims, and drawings associated with the current application. The phrases “in one embodiment,” “in another embodiment,” “in various embodiments,” “in some embodiments,” “in other embodiments,” and other variations thereof refer to one or more features, structures, functions, limitations, or characteristics of the present disclosure, and are not limited to the same or different embodiments unless the context clearly dictates otherwise. As used herein, the term “or” is an inclusive “or” operator, and is equivalent to the phrases “A or B, or both” or “A or B or C, or any combination thereof,” and lists with additional elements are similarly treated. The term “based on” is not exclusive and allows for being based on additional features, functions, aspects, or limitations not described, unless the context clearly dictates otherwise. In addition, throughout the specification, the meaning of “a,” “an,” and “the” include singular and plural references.

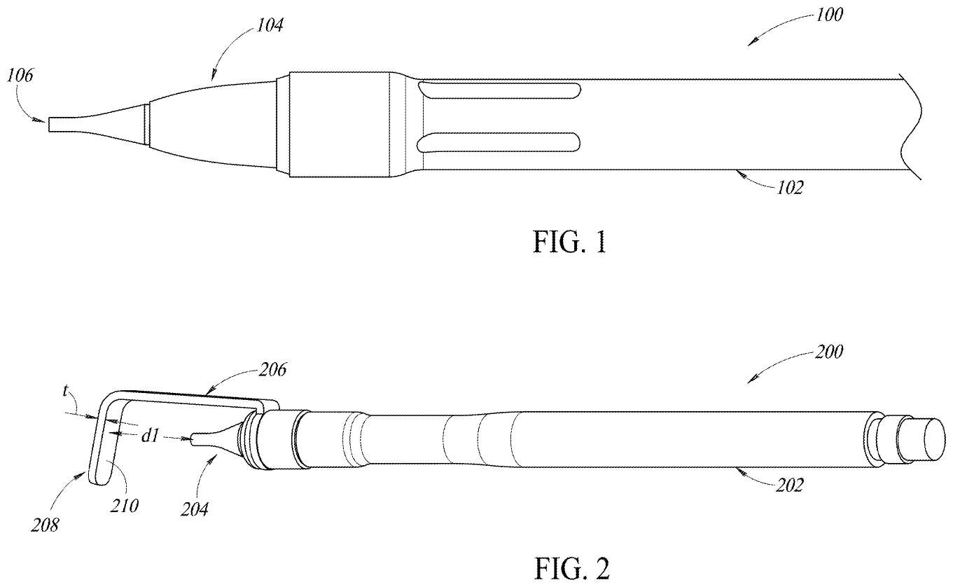

FIG. 1 shows a handpiece 100 for applying a laser to bodily tissue. The handpiece 100 includes an elongated body 102 for gripping and a nozzle 104 having an aperture 106 for emitting a laser onto bodily tissue. Although the handpiece 100 may be useful for some surgical and medical applications, the laser emitted from the aperture 106 is unimpeded and may unintentionally damage bodily tissue in certain situations. For example, in an uvulectomy, some or all of a uvula may be removed using the handpiece 100 to alleviate sleep apnea. However, use of the handpiece 100 in an uvulectomy involves a risk of damage to bodily tissue in the throat and back of the mouth.

FIG. 2 shows a handpiece 200 for applying a laser to bodily tissue in uvulectomy procedures and other surgical procedures. Similar to the handpiece 100, the handpiece 200 includes a body 202 and a nozzle 204 having an aperture 205 from which laser light may be omitted. The handpiece 200 additionally includes a guard 206 attached to the body 202 and having a backstop 208 spaced apart at a distance d1 from an end of the nozzle 204. The backstop 208 is a sheet having a rectangular cross-sectional area and a thin thickness t relative to its length l1. The backstop 208 opposing the nozzle 204 has a flat surface 210 along its width w1. The surface 210 is manufactured with a coarse or rough finish (e.g. sand-blasted) in order to cause the diffuse reflection, and is intended to prevent laser light from unintentionally damaging tissue other than targeted tissue.

The distance d1 of the surface 210 to the end of the nozzle 204 is half an inch or greater. As a result of one or more dimensions associated with the guard 206 (e.g., the distance d1), it may be undesirable, impossible, or uncomfortable to the patient to use the handpiece 200 in certain procedures. The width w1 of the backstop 208 is also similar to the width of the body 202 of the handpiece 200, making it difficult to perform operations in tightly confined or high-risk areas or that involve bodily tissue requiring relatively higher degrees of precision. Moreover, the angle of the surface 210 relative to the optical axis of the laser light is not tightly controlled due to the construction of the guard 206 and due to highly diffused reflected light from surface 210. The shape of the guard 206 also presents other problems related to safety and/or hygiene, as described below.

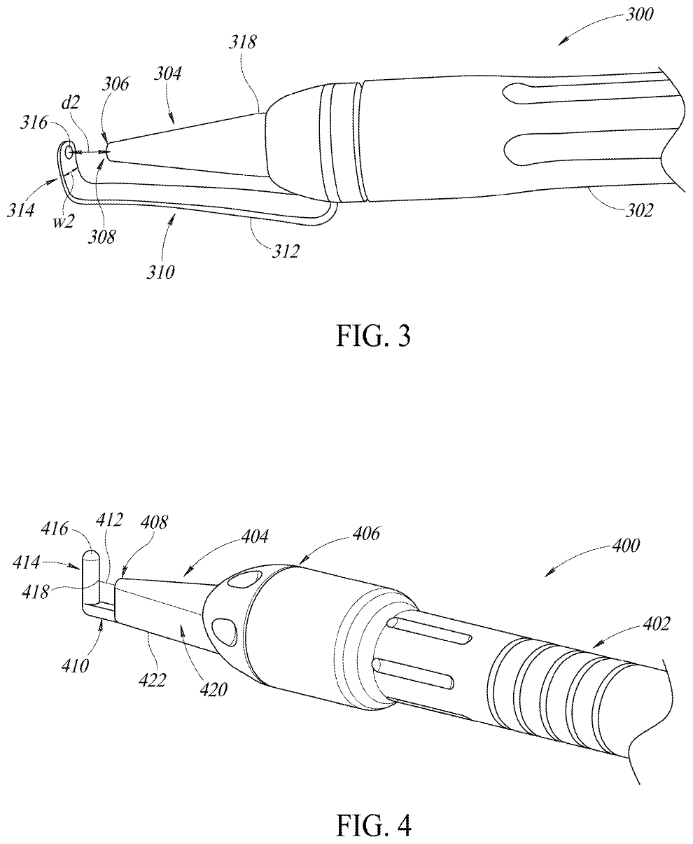

FIG. 3 shows a surgical device 300 according to one or more embodiments for implementing laser light to perform particular surgical and medical procedures. The surgical device 300 includes a body 302 having an elongated shape to facilitate gripping by a user and a nozzle 304 having a frustoconical shape that tapers in thickness toward an end 306 thereof. The end 306 of the nozzle 304 includes an aperture 308 from which laser light can be emitted from the surgical device 300 along an optical axis. Aperture 308 is substantially larger that the size of the laser beam emitted through such aperture.

The surgical device 300 also includes a backstop portion 310 that restricts a distance that the laser light emitted from the aperture 308 can travel without being reflected or diffused.

The backstop portion 310 includes an arm 312 extending from the body portion 302 in a direction along the length of the nozzle 304. A backstop 314 extends from a distal end of the arm 312 toward and intersecting with the optical axis of the laser light. The backstop 314 has a surface 316 located along the optical axis of the laser light. The surface 316 is normal or orthogonal to the optical axis and is spaced apart at a distance d2 from the end 306. The surface 316 may be treated by a process to increase its reflectivity, such as by blasting the surface 316 with an abrasive material (e.g., sand, silicone) or peening the surface 316 (e.g., laser peening, shot peening, ice peening). Alternatively, the surface 316 can be made flat and of high quality surface finish, and to be made to be oriented perpendicular to the axis of the laser beam propagating from the aperture 308. The increased reflectivity of the surface 316 facilitates laser light incident thereupon to be reflected back into the aperture 308 of the surgical device 300 along the optical axis instead of unintentional redirection of the laser light onto bodily tissue.

The distance d2 between the surface 316 and the end 306 is, in at least some embodiments, 4 mm or less. In one embodiment, the distance is approximately twice a focal length of a laser beam emitted from the aperture 308, which also facilitates reflecting laser light from the backstop into the aperture 308. The width W-2 of the backstop 314 is, in at least some embodiments, the same as or less than a cross-sectional size of the end 306. The width w2 is less than a widest portion 318 of the nozzle 304. The reduced size of the backstop 314 relative to the width of the surface to 10 allows for the surgical device 300 to be maneuvered in sensitive areas of bodily tissue or in cavities having a small volume and helps to reduce the risk of unintentional damage to bodily tissue.

In some embodiments, the nozzle 304 and the backstop portion 310 are a single integral piece that is selectively attachable and removable from the body 302. Other nozzle portions having different geometries may be selectively attached and removed from the body 302 for performance of different surgical procedures or performance of surgical procedures in different areas of a body.

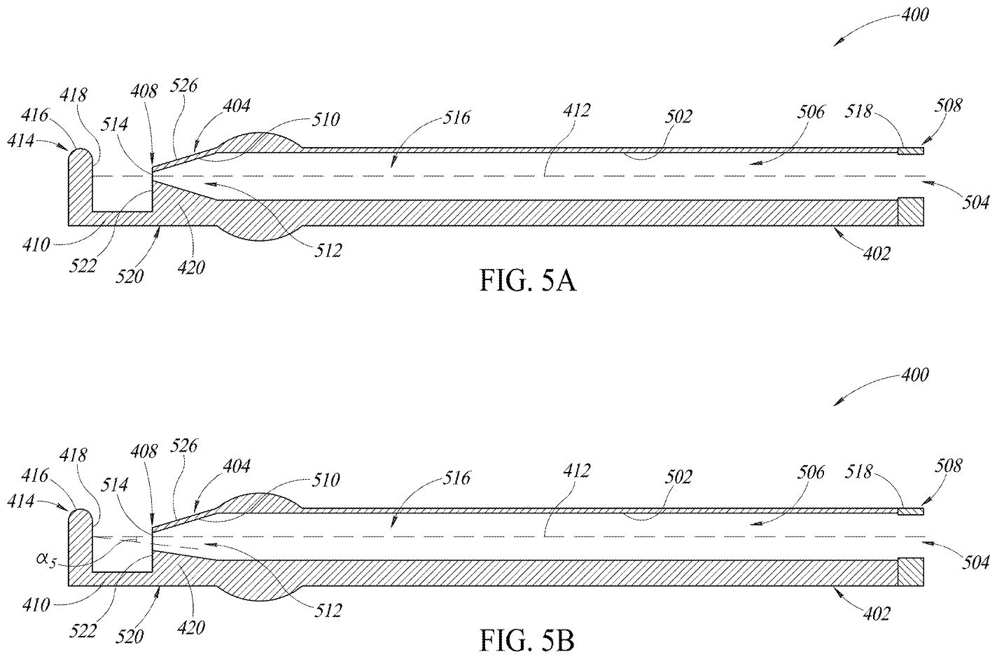

FIG. 4 shows a surgical device 400 according to one or more embodiments for implementing laser light perform particular surgical and medical procedures. The surgical device 400 includes a body 402 having an elongated shape which may have features on a surface thereof to facilitate gripping by user. The surgical device 400 also includes a nozzle portion 404 at a first end of the body 402 that has at least an upper portion with a semi-frustoconical shape that tapers from the first end 406 toward an end 408 of the nozzle portion 404. The end 408 of the nozzle portion 404 includes an aperture (see aperture 514 of FIG. 5) through which laser light is emitted along an optical axis 412. The apertures 408 and 514 are substantially larger that the size of the laser beam emitted through such aperture. The surgical device 400 may also include an arm 410 extending outwardly from the end of the nozzle portion and being peripherally spaced apart from the optical axis 412.

A backstop 414 extends from the arm 410 toward and intersects with the optical axis 412. The backstop 414 shown in FIG. 4 has a cylindrical shape with a circular cross-section that terminates at an upper portion 416 which has a rounded or hemispherical shape that facilitates preventing the backstop 414 from unintentionally poking or piercing bodily tissue of the patient. The optical axis 412 intersects with a rounded wall or surface 418 of the backstop 414. The distance between the end 408 and the surface 418 is, in at least some embodiments, 4 mm or less. This reduced distance enables surgeons to precisely cut, using lasers, bodily tissue having a small thickness or cut bodily tissue located in a small volume or area without risk of unintentional damage to surrounding tissue. In an embodiment, the distance is approximately twice the depth of focus near the focal plane (focal plane is located within the space between the end of the nozzle 408 and the backstop 414) of a laser beam emitted from the aperture 514, which facilitates reducing the risk of laser light escaping an unintentionally damaging tissue.

In one embodiment, the surface 418 may be treated by abrasive blasting or peening, as described above, to cause diffuse reflectivity of the surface 418. The rounded sidewall surface 418 has a rounded or curved surface area from which the laser light is diffused. In particular, the laser light is diffused in different directions each normal to a location of the surface 418 at which the laser light is incident upon. Diffusing the laser light causes the rays of laser light to diverge in numerous directions, thereby reducing power of the laser light and reducing or eliminating risk of damage to surrounding bodily tissue due to reflected laser light.

The nozzle portion 404 also includes a wall 420 extending from the end 408 to the first end 406 along a length of the nozzle portion 404 in a direction along the optical axis 412. In some embodiments, the wall 420 has a thickness extending between and corresponding to lower sides of the nozzle portion 404. The wall 420 may taper in thickness from the first end 406 to the end 408. The wall 420 promotes hygiene and cleanliness by eliminating the space between the nozzle and the arm that could potentially collect bodily tissue and/or fluid or harbor pathogens.

The nozzle portion 404 and backstop 414 have rounded surfaces, such as the upper portion 416 and the frustoconical shape of 404, and/or a peripherally facing surface 422 of the arm 410. The rounded surfaces of the surgical device 400 help to reduce or prevent scraping or irritating bodily tissue that may occur as a result of contact between the surgical device 400 and bodily tissue.

In an embodiment, the backstop 414 instead of having a cylindrical surface 418 may have a surface similar to the surface 316 of FIG. 3, and may be normal or orthogonal to the optical axis 412. The surface 418 may be treated by a process to increase its reflectivity. The increased reflectivity of the surface 418 facilitates laser light incident thereupon to be reflected back into the aperture (see aperture 514 of FIG. 5) of the surgical device 400 along the optical axis instead of unintentional redirection of the laser light onto bodily tissue. The distance between the surface 418 and the end 408 is, in at least some embodiments, 4 mm or less. In an embodiment, the distance is approximately twice the depth of focus near the focal plane (focal plane is located within the space between the end of the nozzle 408 and the backstop 414) of a laser beam emitted from the end 408, which also facilitates reflecting laser light from the backstop into the aperture.

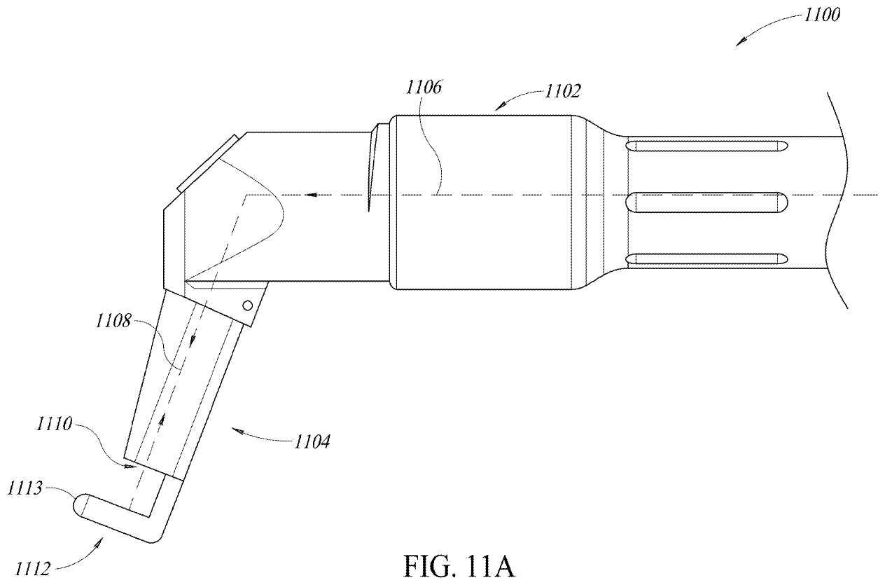

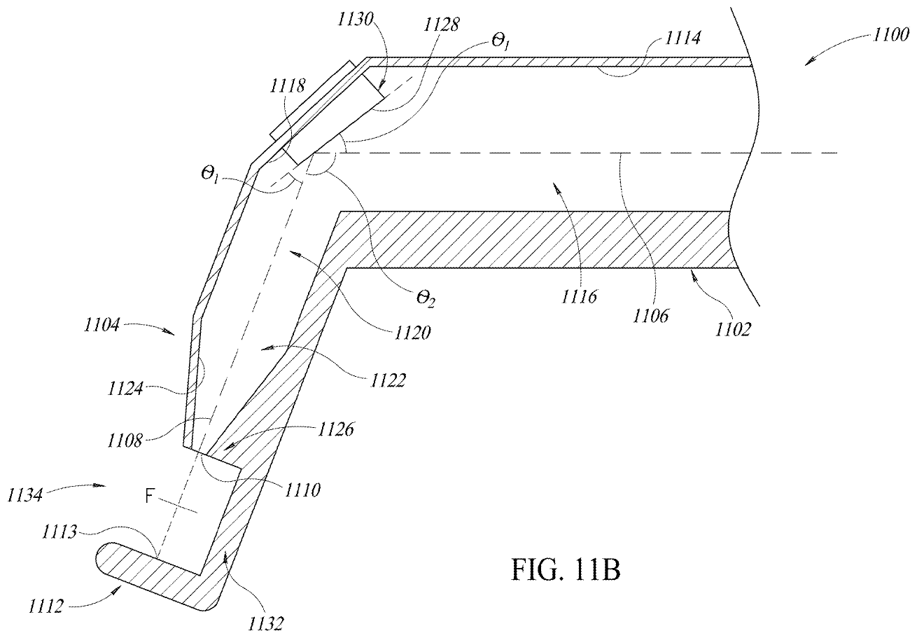

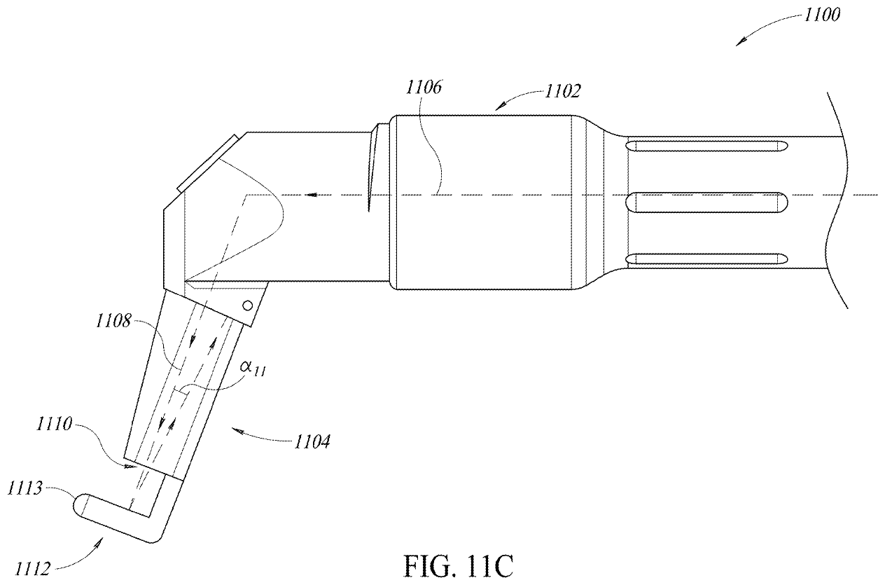

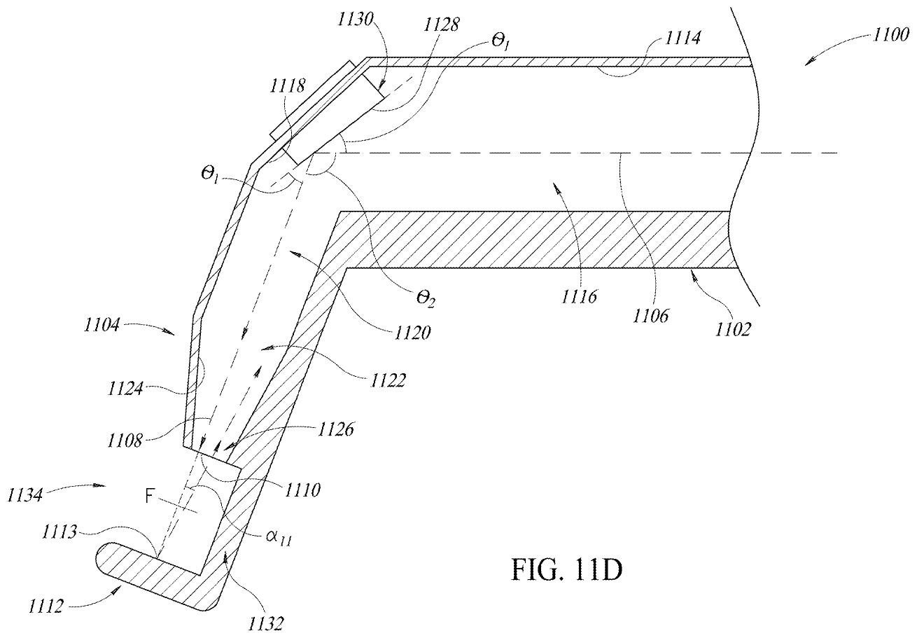

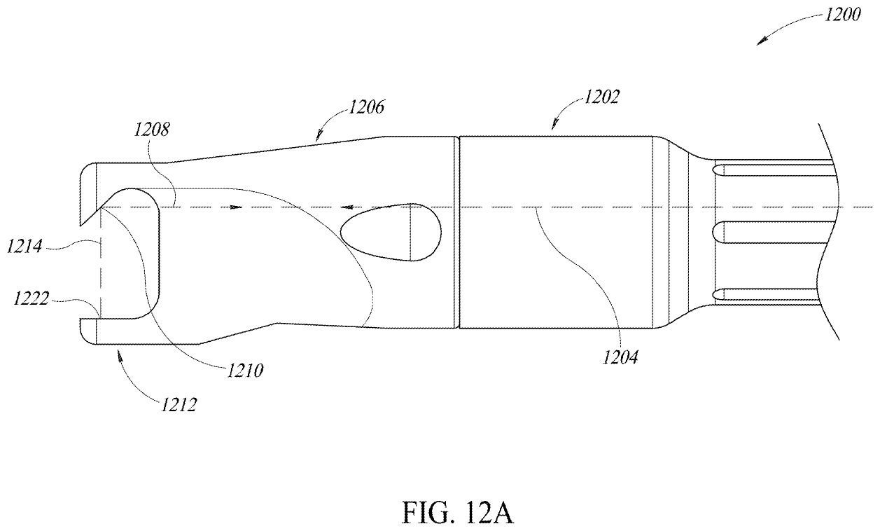

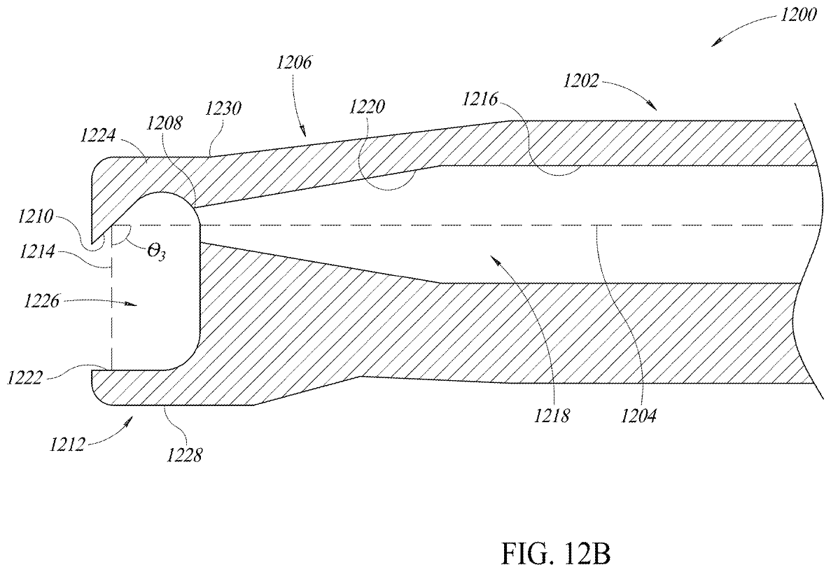

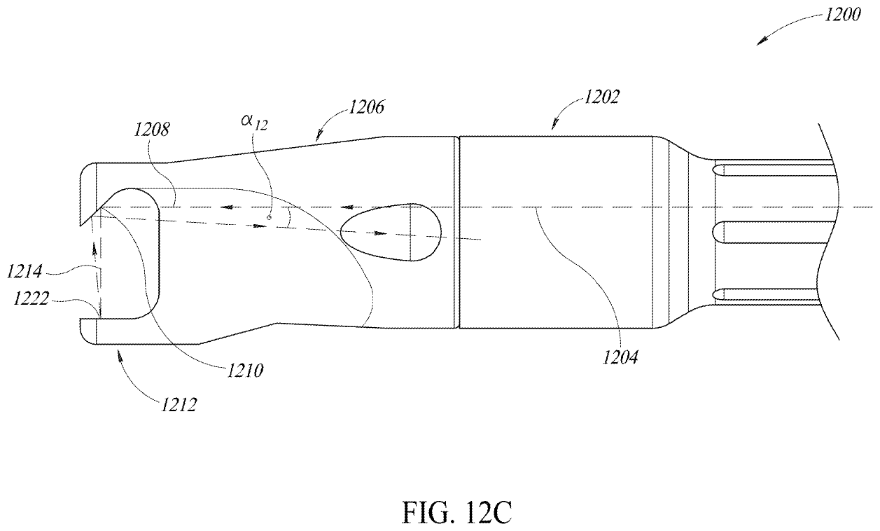

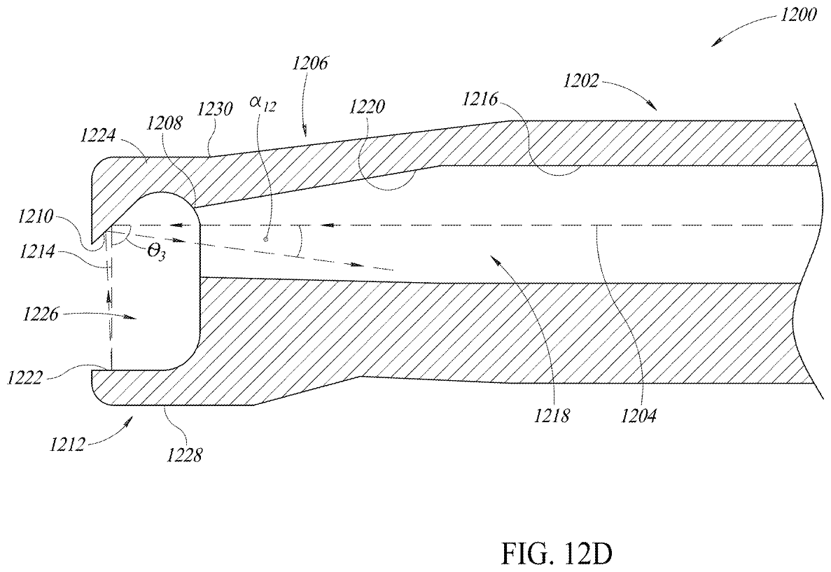

The nozzle portion 404 and the body 402 are configured for selective attachment and/or removal of the nozzle portion 404 from the body 402. Other nozzle portions described herein may be selectively attached to the body 402, such as those described below with respect to FIGS. 11A, 11B, 12A, and 12B.

FIG. 5A shows a longitudinal cross-sectional view taken along a length of the surgical device 400 according to one or more embodiments. The longitudinal cross-sectional view shows an interior of the surgical device 400 taken along the optical axis 412. The body of the surgical device 400 has interior sidewalls 502 extending in parallel with each other from an aperture 504 along the optical axis 412 to define a first cavity portion 506. The body 402 of the surgical device 400 may have interior conical sidewalls 502 extending in a converging fashion with each other from an aperture 504 along the optical axis 412 to define a first cavity portion 506. The aperture 504 is located at an end 508 of the body 402 opposite to the nozzle portion 404. The aperture 504 is sized and shaped for insertion of a laser beam transmission member, such as a fiber tip of a fiber waveguide for emitting laser light from a laser beam emission source of a laser beam delivery system. The laser beam transmission member may be an end of an articulated arm that includes a laser light source (e.g., laser diode, solid-state laser). The first cavity portion 506 is sized and shaped to receive a length of the fiber waveguide, as described herein.

The nozzle portion 404 has interior sidewalls 510 that are oriented at an acute angle with respect to the optical axis 412 to define a second cavity portion 512, which tapers in cross-sectional area toward the end 408. The end 408 of the nozzle portion 404 includes an aperture 514 defining a terminus of the second cavity portion 512. The aperture 514 is substantially larger that the size of the laser beam emitted through such aperture. The optical axis 412 extends through the center of the aperture 514 and through at least a portion of the second cavity portion 512. The first cavity portion 506 and the second cavity portion 512 collectively define a cavity 516 of the surgical device 400. The cavity 516 may include additional support structure for guiding the fiber waveguide therethrough and restricting movement and/or vibration of the fiber waveguide therein. However, such support structures are omitted from the Figures for clarity.

An attachment portion may be provided at the end 508 to selectively attach and secure the fiber waveguide to the cavity 516. The attachment portion may engage with a corresponding attachment portion on the fiber waveguide for attachment. Non-limiting examples of the attachment portions include a threaded surface on exterior walls 518 of the body 402, a threaded surface on the interior walls 502 at the end 508, magnetic elements at the end 508, fasteners at or near the end 508 (e.g., within the first cavity portion 506, on the exterior walls 518) that engage with corresponding fasteners of the fiber waveguide. Other attachment portions known to those of ordinary skill in the art are considered as being within the scope of the present disclosure.

The wall 420 extends from the second cavity portion 512 to a lower exterior side 520 of the nozzle portion 404. The wall 420 also includes an end or edge 522 extending between the aperture 514 and the arm 410. The end 522 shown in FIG. 5 extends vertically in a direction orthogonal to the optical axis 412; however, the end 522 may extend at an angle with respect to the optical axis 412 in some embodiments. For example, the end 522 may extend at an angle with respect to the optical axis 412 between the aperture 514 and a proximate or lower portion 524 of the backstop 414. In such embodiments, the arm 410 may be considered as being coextensive with the end 522. The wall 420 may help to prevent or reduce collection of bodily fluid, bodily tissue, and/or pathogens, thereby improving the hygiene of the surgical device 400.

The interior sidewalls 510 shown in FIG. 5 extend in parallel with an upper exterior side 526 of the nozzle portion 404. The upper exterior side 526 forms a frustoconical shape ending at the first end 406, which allows a user to access cavities and or bodily tissue having relatively smaller dimensions than those described above with respect to FIGS. 1 and 2. In some embodiments, however, the nozzle portion 404 may have an upper exterior side 526 with a different shape, such as a cylindrical shape with a constant peripheral dimension along its length.

The body 402, the nozzle portion 404, the arm 410, and the backstop 414 of the surgical device 400 shown and described with respect to FIGS. 4 and 5 (and elsewhere herein) are formed of the same material comprising a single monolithic structure. In some embodiments, some of the body 402, the nozzle portion 404, the arm 410, and the backstop 414 may be different parts that are assembled to form the surgical device 400 shown in FIG. 4. For example, the nozzle portion 404, the arm 410, and the backstop 414 may be a first monolithically formed part and the body 402 may be a second monolithically formed part. The first monolithically formed part and the second monolithically formed part may be assembled to form the surgical device 400. The first monolithically formed part may include a first attachment portion and the second monolithically formed part may include a second attachment portion corresponding to the first attachment portion. The first attachment portion may be engaged with the second attachment portion to assemble the surgical device 400. Non-limiting examples of the first attachment portion and the second attachment portion include threaded surfaces, fasteners, and interlocking portions that engage to secure the first attachment portion and the second attachment portion.

FIG. 5B shows a longitudinal cross-sectional view taken along a length of the surgical device 400 according to one or more embodiments. In the cross-sectional view of the surgical device 400 of FIG. 5B, laser light directed along the axis 412 (e.g., travelling parallel to the axis 412, centered along the axis 412) is incident upon the backstop 414. The backstop 414 reflects the laser beam at an acute angle α₅ relative to the axis 412. The reflected laser light enters the aperture 504 and travels into the cavity 516. The laser light reflected back into the cavity 516 may reduce the likelihood that the laser light will unintentionally damage surrounding bodily tissue.

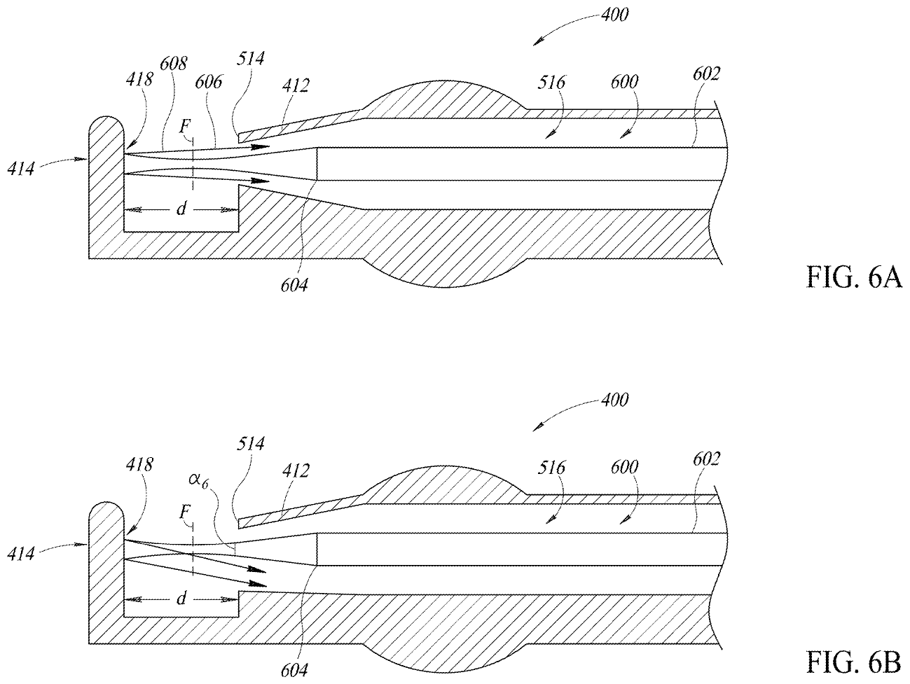

FIG. 6A shows the longitudinal cross-sectional view taken along a length of the surgical device 400 with a fiber waveguide 600 according to one or more embodiments. The fiber waveguide 600 has a waveguide body 602 having an elongated cylindrical shape that extends at least partially through the cavity 516. The waveguide body 602 is typically a rigid member, but may be flexible or bendable along its length in some embodiments. The fiber waveguide 600 may include an optical core with a high refractive index for transmitting or carrying an optical signal and cladding having a lower refractive index relative to the optical core for confining the optical signal in the optical core. Alternatively, the fiber waveguide 600 may be designed as a hollow core waveguide with low refractive index of the core and higher refractive index on the walls of such hollow waveguide. Some portions of the fiber waveguide 600 not shown may include a cover for protecting the optical core and the cladding therein.

In one embodiment, the fiber waveguide 600 may include a focusing lens located between the fiber end and the aperture 514. The focusing lens helps to facilitate a better fit of the laser beam through the aperture 514.

In another embodiment, an end of the fiber waveguide 600 may be proximate or adjacent to the aperture 514. In such cases, it may unnecessary to include the focusing lens in the fiber waveguide 600.

In another embodiment, when an articulated arm is attached to the device at the proximal end 508, a focusing lens may be placed inside the cavity 506 to facilitate a better fit of the laser beam through the aperture 514.

The laser light 606 is emitted from an end 604 of the fiber waveguide 600 and travels through the aperture 514, and to the surface 418 of the backstop 414. Bodily tissue present between the aperture 514 and the surface 418 may be destroyed (e.g., evaporated, incinerated) by the laser light 606 incident thereupon. The laser light 606 is focused at a focal distance F located between the surface 418 and the aperture 514. The surface 418 of the backstop 414 is spaced apart from the aperture 514 at a distance of d3, which is 6 mm or less in at least some embodiments, which facilitates laser light from unintentionally damaging bodily tissue in an area of the body, such as the mouth, other than the target bodily tissue. The focal plane distance F is located approximately at a point that is half of the distance d3 between the surface 418 and the aperture 514. Therefore, the point of the laser light 606 that is the strongest is located about halfway between the surface 418 and the aperture 514. Furthermore, the depth of focus near the focal plane (focal plane is located within the space between the end of the nozzle aperture 514 and the backstop surface 418) is approximately half the distance between the nozzle aperture 514 and the backstop surface 418.

The fiber waveguide 600 and/or the surgical device 400 may include features for ensuring that the end 604 is appropriately located to position the focal distance F of the laser light 606 halfway between the surface 418 and the aperture 514 (i.e., half of d3). Such features may include corresponding attachment portions of the fiber waveguide 600 and the surgical device 400.

FIG. 6B shows a longitudinal cross-sectional view taken along a length of the surgical device 400 according to one or more embodiments. In the cross-sectional view of the surgical device 400 shown in FIG. 6B, laser light directed along the axis 412 is incident upon the backstop 414. The backstop 414 reflects at least some of the laser light at an angle α₆ and back into the cavity 516 through the aperture 514.

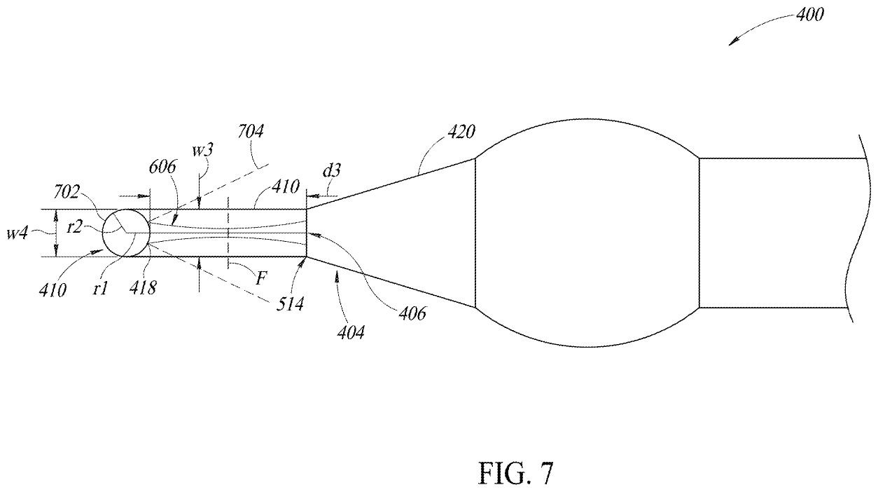

FIG. 7 shows a top plan view of the surgical device 400 emitting laser light 606 from the fiber waveguide 600 according to one or more embodiments. As discussed above with respect to FIG. 6 and elsewhere, the laser light 606 is emitted from the aperture 514 and propagates along the optical axis 412 and is incident upon the surface 418. The arm 410 has a width w3 that is approximately equal to or less than a width of the end 406 of the nozzle portion 404. A width w4 of the backstop 414 is approximately equal to or less than the width w3 of the arm 410. The surface 418 of the backstop opposing the aperture 414 has a curved or rounded surface with a radius of curvature r1 about an axis of the backstop 414. Some or all of the surface 418 may be treated (e.g., blasted, peened, polished) to improve or adjust its diffuse reflectivity. The radius r1, in some embodiments may be half of the width w4 such that the backstop 414 has at least a semicircular cross-sectional shape in the view shown in FIG. 7. The laser light 606 is incident upon a portion of the curved or rounded surface 518 that diffuses rays of the laser light 606 in different directions, thereby forming defocused or scattered laser light 704 as a result of incidence upon the surface 518. The scattered laser light 704 is not powerful enough to destroy or damage surrounding bodily tissue.

The backstop 414 has a surface 702 on a side of the backstop 414 opposite to the surface 518. The surface 702 may also have a radius of curvature r2 about the axis of the backstop 414. The radius r2, in some embodiments, may be the same as the radius r1 such that the backstop 414 has a circular cross-sectional shape in the view presented in FIG. 7. However, in some embodiments, the radius r2 may be different than the radius r1 such that the backstop 414 has an asymmetric cross-sectional shape in the view presented in FIG. 7. The curved or rounded surface 702 may be used for blunt dissection of bodily tissue but may facilitate avoiding unintentionally irritating, scratching, or cutting bodily tissue in other situations. In some embodiments, the surface 702 may be wider than the width w3.

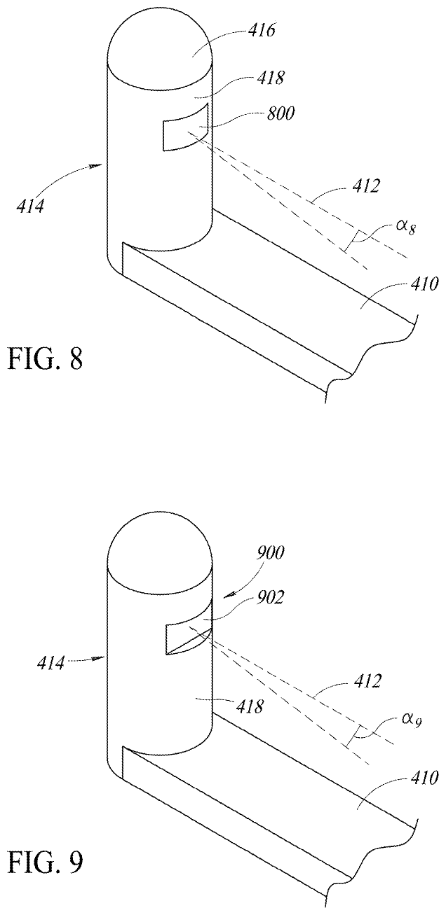

FIG. 8 shows an overhead perspective view of the backstop 414 and the arm 410 according to one or more embodiments. The surface 418 of the backstop 414 has a portion 800 that is treated to improve or adjust the diffuse reflectivity of the portion 800. Treating the portion 800 may include blasting the portion 800 of the surface 418 with an abrasive material (e.g., sand, silicone), peening the portion 800 (e.g., laser peening, shot peening, ice peening), or polishing the portion 800. The portion 800 has the same radius r1 as the rest of the surface 418. The portion 800 is located along the optical axis 412 such that the laser light 606 incident upon the portion 800 is diffused as the scattered light 704. The portion 800 of the surface 412 reflects at least some laser light traveling along the axis 412 (e.g., parallel to the axis 412, centered along the axis 412) at an angle α₈ relative to the axis 412. The laser light reflected at the angle α₈ may travel back into the cavity of the surgical device 400.

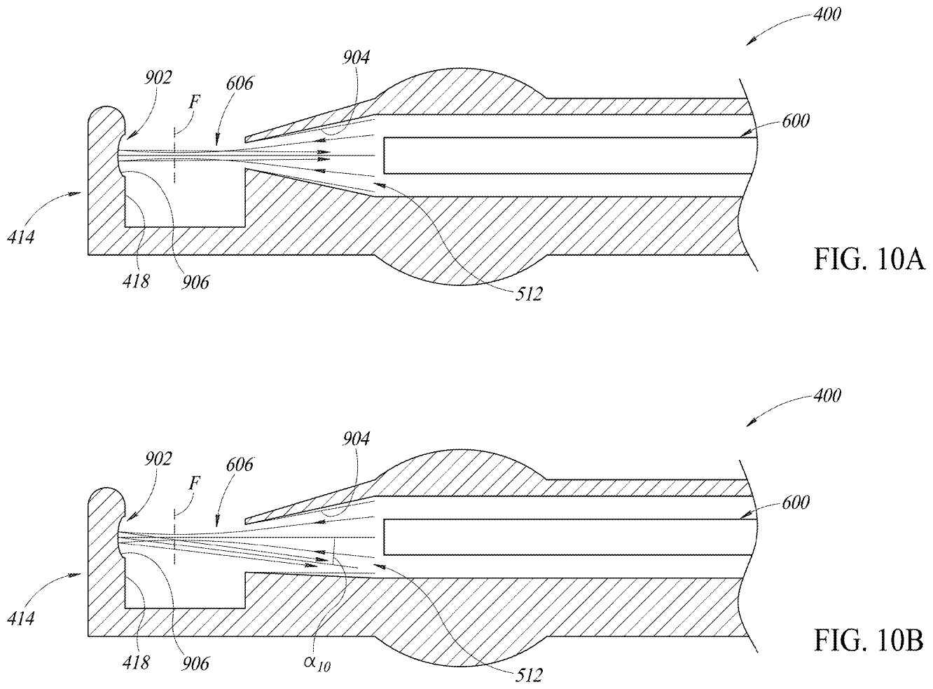

FIG. 9 shows an overhead perspective view of the backstop 414 and the arm 410 according to one or more embodiments. The surface 418 of the backstop 414 has a recess 900 receding relative to the radius of curvature r1 of the surface 418. The recess 900 may be formed as a result of milling, ablating, broaching, or otherwise removing material from the backstop 414; or may be formed as a result of a casting process to form the backstop 414. The recess 900 may extend for the most of the height, or the whole height of the backstop 414. The recess 900 includes a flat surface 902 therein that is located along the optical axis 412. In particular, the flat surface 902 is orthogonal to the optical axis 412. The flat surface 902 may be treated to improve or adjust its mirror like reflectivity, through processes like polishing or high surface finish machining and or through coating the surface with the high reflectivity materials such as gold. The laser light 606 incident upon the flat surface 902 is reflected back into the second cavity portion 512 through the aperture 514, reducing the risk of unintentional damage to bodily tissue around the bodily tissue targeted for operation. The flat surface 902 of the recess 900 reflects at least some laser light traveling along the axis 412 (e.g., parallel to the axis 412, centered along the axis 412) at an angle α₉ relative to the axis 412. The laser light reflected at the angle α₉ may travel back into the cavity of the surgical device 400. FIG. 10A shows a longitudinal cross-sectional view taken along a length of the surgical device 400 according to one or more embodiments. The backstop 414 of the surgical device has a concave portion 902 opposing the aperture 514 and located along the optical axis 412 of the laser light 606 to be emitted by the fiber waveguide 600. The concave portion 902 is curved inwardly with respect to a vertically extending surface 418 of the backstop 404. The concave portion 902 is also curved inwardly with respect to the radius of curvature r1 of the backstop 414. The concave portion 902 may have a symmetric shape centered on the optical axis 412. The surface of the concave portion 906 may be treated to improve or adjust its reflectivity, as described herein. The laser light 606 incident upon the surface 906 is reflected back into the second cavity portion 512 as diffused or scattered light 904. As a result, the risk of unintentional damage to bodily tissue near or around the bodily tissue targeted for operation is reduced.

FIG. 10B shows a longitudinal cross-sectional view taken along a length of the surgical device 400 according to one or more embodiments. In the cross-sectional view of FIG. 10B, laser light travelling along the optical axis 412 is emitted from the aperture 512 and is incident upon the concave portion 902. The concave portion 902 reflects the laser light at angle am and back into the second cavity portion 512 via the aperture 514.

FIG. 11A shows a surgical device 1100 according to one or more embodiments. The surgical device 1100 includes a body 1102 having an elongated shape extending along a first axis 1106. The surgical device 1100 includes a nozzle portion 1104 that is oriented at an angle with respect to the first axis 1106. The surgical device 1100 includes a nozzle portion 1104 that is oriented at an angle with respect to the first axis 1106. In particular, the nozzle portion 1104 extends along a second axis 1108 that is oriented at an oblique or obtuse angle relative to the first axis 1106. The nozzle portion 1104 includes an aperture 1110 from which laser light may be emitted toward a backstop 1112. The backstop 1112 may have a reflective region opposing the aperture 1110 and located along the second axis 1108. The reflective region may be treated to adjust its reflectivity to diffuse or reflect laser light, as described with respect to FIGS. 8 and 9 and elsewhere herein. The backstop 1112 may have a rounded cross-sectional shape (e.g., circular, rectangular with rounded edges) and a rounded upper end 1113 to reduce the risk of damaging or snagging bodily tissue.

FIG. 11B shows a longitudinal cross-sectional view taken along a length of the surgical device 1100 according to one or more embodiments. The body 1102 includes first inner sidewalls 1114 extending along the first axis 1106 to define a first cavity portion 1116. The nozzle portion 1104 includes second inner sidewalls 1118 extending along the second axis 1108 to define a second cavity portion 1120. The nozzle portion 1104 further includes a tapered portion 1122 with third inner sidewalls 1124 that taper inwardly from the second cavity portion 1120 toward the aperture 1110 at an end 1126 of the nozzle portion 1104. The surgical device 1100 may be used to perform certain operations for which it may be desirable to cut at an angle. The backstop 1112 is spaced apart from the end 1126 at a distance of 6 mm or less, which facilitates laser light from unintentionally damaging bodily tissue in an area of the body, such as the mouth, other than the target bodily tissue. The backstop 1112 may include a reflective surface 1115 opposing the aperture 1110 along the second axis 1108. The reflective surface 1115 may be treated to adjust reflectivity thereof as described elsewhere herein. In some embodiments, the reflective surface 1115 may be flat and laser light incident thereupon is reflected at least partially back into the nozzle portion 1104.

The nozzle portion 1104 and body 1102 may be configured for selective attachment and removal of the nozzle portion 1104 to and from the body 1102. Other nozzle portions may then be attached to the body 1102, such as the nozzle portion 404 or the nozzle portion 1206 described below with respect to FIGS. 12A and 12B.

The surgical device 1100 includes an optical element provided within the first cavity portion 1116 and/or the second cavity portion 1120. The optical element shown in FIG. 11B is a reflective surface 1128 provided within the first cavity portion 1116 and/or the second cavity portion 1120. The reflective surface 1128 is positioned and oriented to receive laser light emitted from a fiber tip of a fiber waveguide and reflect the incident light at least partially along the second axis 1108. For instance, at the intersection of the first axis 1106 and the second axis 1108, the reflective surface 1128 may be oriented at an oblique angle θ₁ with respect to the first axis 1106 and oriented at the same oblique angle θ₁ with respect to the second axis 1108. An angle θ₂ between the first axis 1106 and the second axis 1108 satisfies the relationship θ₂=180°−(2*θ₁), where θ is in degrees.

In the embodiment shown, the reflective surface 1128 is flat. In some embodiments, however, the reflective surface 1128 may be concave and have a curvature designed to focus the laser light at a focal point F between the backstop 1112 and the end 1126. In some embodiments, the reflective surface 1128 may have a radial curvature similar to a curvature of the inner sidewalls of the surgical device 1100.

The reflective surface 1128 may be achieved by treating the surface by blasting the surface with an abrasive material, peening the surface, polishing the surface, or any other suitable method for increasing or adjusting reflectivity of the surface. The reflective surface 1128 may be located on a platform 1130 or other such structure projecting toward the first axis 1106 and/or the second axis 1108 from an inner sidewall of the surgical device 1100. In some embodiments, the platform 1130 may extend from the second inner sidewalls 1118 and not the first inner sidewalls 1114, which may facilitate selective attachment and removal of the nozzle portion 1104 from the body 1102. Other nozzle portions may then be attached to the body 1102, such as the nozzle portion 404 or the nozzle portion 1206 described below with respect to FIGS. 12A and 12B. In some embodiments, the platform 1130 may be part of a plug that is installed in a receptacle on the nozzle portion 1104 or the body 1102.

In some embodiments, the optical element may be an optical waveguide instead of the reflective surface 1128. The optical waveguide may receive laser light from a fiber waveguide inserted in the body 1102 and direct the laser light through the aperture 1110 onto the backstop 1112.

The surgical device 1100 includes an arm 1132 connecting the backstop 1112 with the nozzle portion 1104. A recess or space 1134 is provided along the arm 1132 between the aperture 1110 and the backstop 1112. An operator may position bodily tissue within the space 1134 for incision, excision, dissection, or other suitable surgical operation thereon. In the embodiment shown in FIGS. 11A and 11B, the space 1134 of the surgical device 1100 opens upwardly in a direction toward the first axis 1106 that forms an acute angle with the second axis 1108. In some embodiments, the backstop 1112 may open downwardly (i.e., away from the first axis 1106) or in a different direction according to the application.

FIG. 11C shows a side view of the surgical device 1100 according to one or more embodiments. Laser light travelling along the second axis 1108 (e.g., laser light that is in parallel with the second axis 1108, laser light that is centered along the second axis 1108) is emitted from the aperture 1110 and is incident upon the backstop 1112. The backstop 1112 reflects at least some of the laser light at an angle α₁₁.

FIG. 11D shows a cross-sectional view of the surgical device 1100 as described with respect to FIG. 11C. At least some of the laser light incident upon surface 1115 of the backstop 1112 is reflected at the angle α₁₁ and enters back into the second cavity portion 1120 through the aperture 1110.

FIG. 12A shows a surgical device 1200 according to one or more embodiments. The surgical device 1200 includes a body 1202 having an elongated shape extending along a first axis 1204 and includes a nozzle portion 1206 also extending, at least in part, along the first axis 1204. The nozzle portion 1206 includes an aperture 1208 located along the first axis 1204 and includes a first reflective surface 1210 located along the first axis 1204 and opposing the aperture 1208. The nozzle portion 1206 further includes a backstop 1212 opposing the first reflective surface 1210 along a second axis 1214 transverse to the first axis 1204. The nozzle portion 1206 and body 1202 are configured for selective attachment and removal of the nozzle portion 1206 to and from the body 1202. Other nozzle portions may then be attached to the body 1102, such as the nozzle portion 404 or the nozzle portion 1206 described below with respect to FIGS. 12A and 12B.

FIG. 12B shows a longitudinal cross-sectional view taken along a length of the surgical device 1200 according to one or more embodiments. The body 1202 includes first inner sidewalls 1216 extending along the first axis 1204 to at least partially define a cavity 1218. The nozzle portion 1206 includes second inner sidewalls 1220 also extending along the first axis 1204 and being angled inwardly toward the first axis 1204 to define a tapered cavity within the surgical device 1200.

The first reflective surface 1210 has high reflectivity to reflect incident laser light from the aperture 1208 toward the backstop 1212. The high reflectivity of the first reflective surface 1210 reflects laser light to maintain an intensity of light that is sufficient to cut bodily tissue. The first reflective surface 1210 may be treated using a process to increase its reflectivity, such as by blasting the with an abrasive material (e.g., sand, silicone), peening the surface (e.g., laser peening, shot peening, ice peening), polishing the surface, or other methods suitable to produce a desired reflectivity. The reflective surface 1210 is positioned to receive laser light emitted from a fiber tip of a fiber waveguide and reflect the incident light at least partially along a second axis 1214.

The first reflective surface 1210 is oriented at an oblique angle with respect to the first axis 1204, the angle selected to reflect incident laser light along the second axis 1214 and onto a second reflective surface 1222 opposing the first reflective surface 1210. As a non-limiting example, the first reflective surface 1210 may be oriented at a 45 degree angle relative to the first axis 1204. As a result, incident laser light from the aperture 1208 is reflected at an angle θ₃ of approximately 90° and along the second axis 1214 toward the second reflective surface. In some embodiments, the first reflective surface 1210 may be oriented at a different angle θ₃ relative to the first axis 1204 and the length of the backstop 1212 and the position of the second reflective surface 1222 along the backstop 1212 may be adjusted to reflect or diffuse the laser light from the first reflective surface.

In the embodiment shown, the first reflective surface 1210 is flat; however, in some embodiments, the reflective surface 1210 may be curved (e.g., concave with respect to the aperture 1208) to focus the laser light at a desired focal distance between the first and second reflective surfaces 1210 and 1222. In the embodiment shown in FIG. 12A, the space between the first reflective surface 1210 and the aperture 1208 is visible when viewed from the side. In some embodiments, however, the space between the first reflective surface 1210 and the aperture 1208 may be occluded or protected to prevent unintentional contact or interference with the laser light beam emitted from the aperture 1208. The second reflective surface 1222 may be convex in some embodiments to diffuse laser light, as described with respect to FIG. 8 and elsewhere herein. In some embodiments, the second reflective surface 1222 may be flat and laser light incident thereupon is reflected back to the first reflective surface 1210, which reflects the laser light back into the cavity 1218, as described with respect to FIG. 9 and elsewhere herein.

The nozzle portion 1206 includes an arm 1224 projecting from the nozzle portion 1206 and near the aperture 1208. A distal end portion of the arm 1224 is equipped with the first reflective surface 1210. A recess or space 1226 is provided between the arm 1224 and the backstop 1212. An operator may position bodily tissue within the recess 1226 for incision, excision, dissection, or other suitable surgical operation thereon. Because the recess 1226 opens outwardly and away from the body 1202, the operator may use sides 1228 and 1230 respectively of the backstop 1212 and the arm 1224 to move or apply pressure to bodily tissue without unintentional application of laser light to such bodily tissue.

FIG. 12C shows a side view of the surgical device 1200 according to one or more embodiments. Laser light travelling along the second axis 1214 (e.g., laser light that is in parallel with the second axis 1214, laser light that is centered along the second axis 1214) is incident upon the second reflective surface 1222. The reflective surface 1222 reflects at least some of the laser light at an angle α₁₂. The reflected laser light is incident upon the first reflective surface 1210.

FIG. 12D shows a cross-sectional view of the surgical device 1200 as described with respect to FIG. 12C. At least some of the laser light incident upon the first reflective surface 1210 is reflected at the angle α₁₃ relative to the first optical axis 1204 and enters back into the cavity 1218 through the aperture 1208.

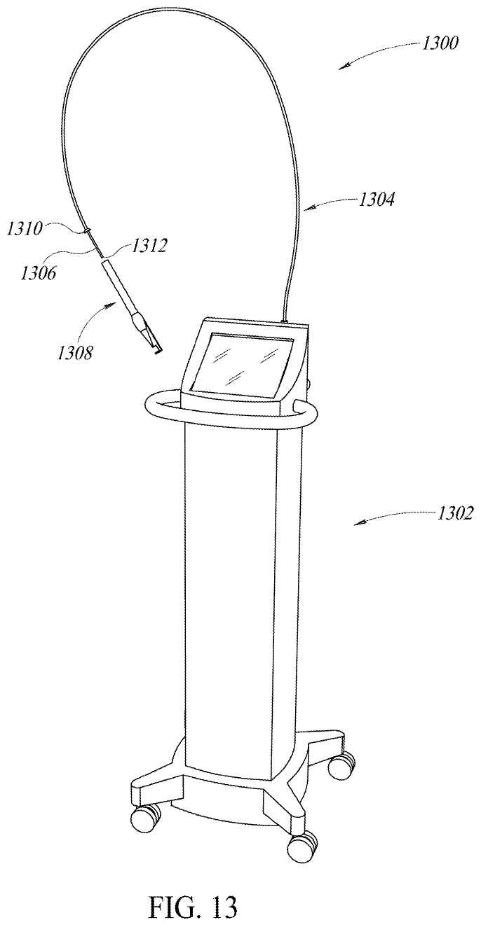

FIG. 13 shows a laser surgery system 1300 according to one or more embodiments. The laser surgery system 1300 includes a laser light source 1302 configured to generate laser light, a fiber waveguide 1304 configured to propagate laser light generated by the laser light source 1302 from one end of the waveguide 1304 to a fiber tip 1306 thereof, and a surgical device 1308 for directing the laser light emitted by the fiber tip 1306 to perform surgical or medical operations. The laser light source 1302 may be a laser light source configured to generate laser light sufficient to destroy (e.g., evaporate, incinerate) certain bodily tissue (e.g., skin, muscle). In some embodiments, the fiber waveguide 1304 may instead be an articulated arm having laser light source at an end portion thereof. An example of the laser light source 1302 is a carbon dioxide laser light source. In an embodiment, the laser light source 1302 is distinguishable from diode laser light sources which are insufficient to directly destroy bodily tissue, such as muscle or skin, and which operate by heating another material (e.g., glass) to a temperature sufficient to burn said tissue. Laser light generated by carbon dioxide laser light sources provides numerous benefits relative to metal surgical instruments and/or diode lasers because the carbon dioxide laser light results in less bleeding, reduced scar tissue, causes less pain, and reduced risk of infection, among others.

In some embodiments, the laser light source 1302 is a diode laser or an neodymium-doped yttrium aluminum garnet (Nd:YAG) laser. In such embodiments, the fiber tip 1306 is a solid core fiber that extends through an aperture of the surgical device 1308. For instance, with reference to FIGS. 4 and 5, waveguide 600 may extend through the aperture 514 and between the surface 418 and the end 408. The target bodily tissue is destroyed when solid fiber core contacts the target bodily tissue.

The fiber waveguide 1304 may include an attachment portion 1310 for selectively attaching the fiber waveguide 1304 to the surgical device 1308 and securing the fiber tip 1306 therein. A lens may be attached to the end of the fiber tip 1306 in order to facilitate the better fit of the laser beam through the aperture of the surgical device 1308 (e.g., aperture 514). The surgical device 1308 corresponds to the surgical devices 300, 400, 1100, 1200, or 1300 according to one or more of the embodiments described herein.

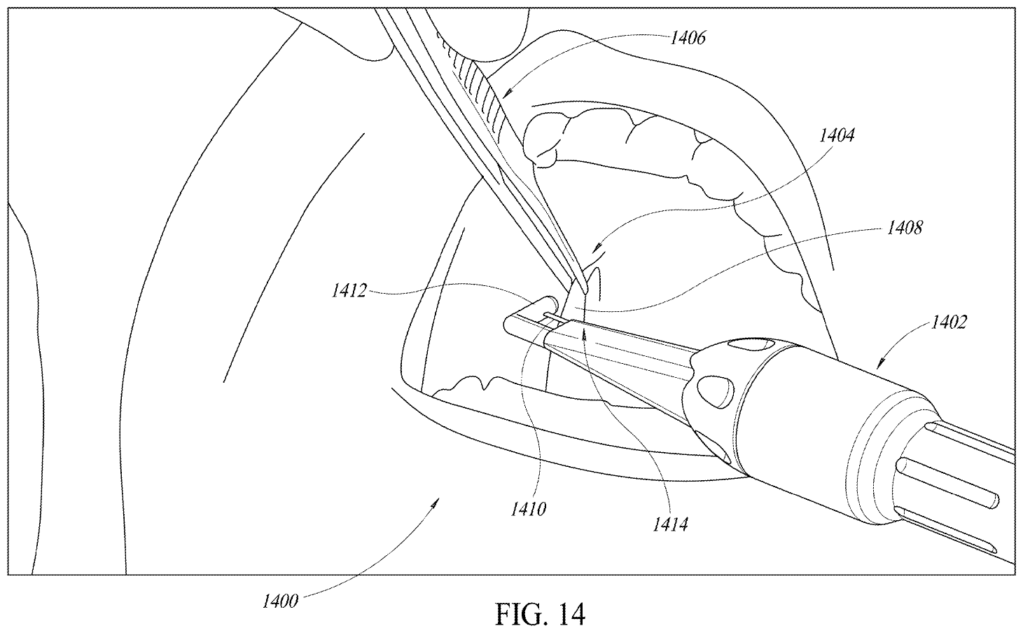

FIG. 14 shows an operation of a surgical procedure 1400 involving a surgical device 1402 according to one or more of the embodiments described herein. The surgical procedure 1400 depicted is known as frenuloplasty in which a frenulum 1404 of a patient is surgically altered or a frenectomy in which at least a portion of the frenulum 1404 is removed. Such procedures may have numerous benefits, such as by alleviating sleep apnea or tension. The procedure 1400 involves gripping the frenulum 1404 with forceps 1406 at the underside of the tongue and making a lateral incision in a medial portion 1408 using laser light 1410 emitted by the surgical device 1402. Advantageously, a backstop 1412, spaced apart at a distance of 6 mm or less from an aperture of a nozzle 1414 of the surgical device 1402, facilitates preventing the laser light 1410 from unintentionally damaging bodily tissue in the mouth other than the desired portions of the frenulum 1404. By contrast, laser light emitted by the handpiece 100 could damage gum tissue or other tissue in the mouth if wielded by a surgeon inexperienced in such procedures. The size and dimensions of the guard 206 of the handpiece 200 are too large and imprecise to be used in the surgical procedure 1400.

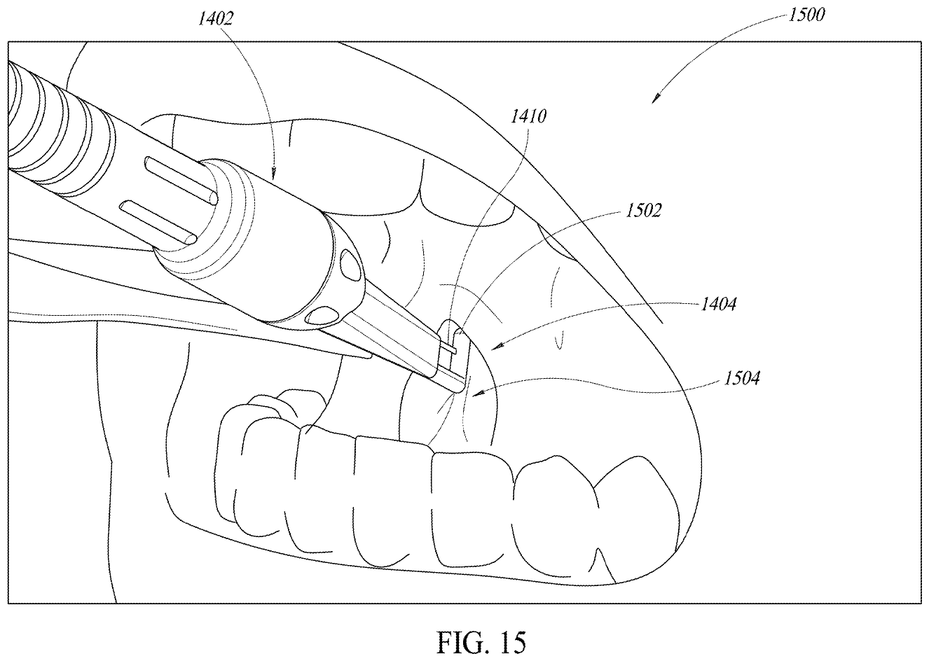

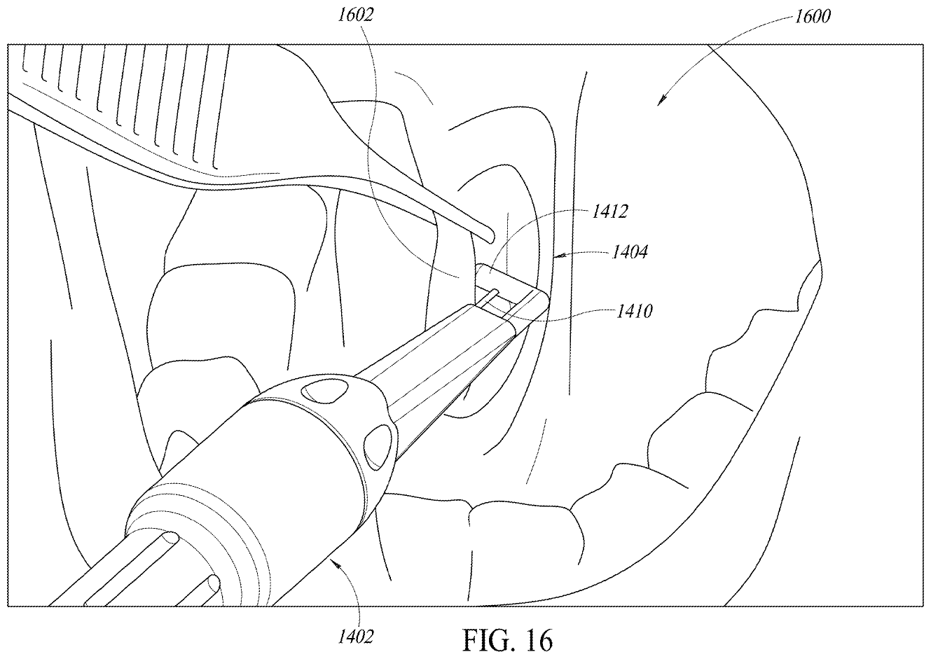

FIG. 15 shows a surgical operation 1500 involved in a frenectomy or frenoplasty procedure using a surgical device described herein according to one or more embodiments. The surgical operation 1500 may be performed subsequent to or in connection with the surgical operation 1400. The surgical operation 1500 involves inserting the backstop 1510 into the lateral incision made in the surgical operation 1400 and making a vertical incision 1502 to vertically bisect the frenulum 1504. Using the handpiece 100 to make the vertical incision 1502 may cause a deeper incision than is necessary and may cause damage to certain bodily tissue, such as nerves or blood vessels. The size and dimensions of the handpiece 200 are too large to perform the vertical incision 1502.

The surgical operation 1500 may also involve blunt dissection of the frenulum by pressing a member, such as a Q-tip or a finger, into a cavity 1504 formed by the vertical incision 151502. Advantageously, the blunt dissection may be performed using the backstop 1410 without potentially introducing pathogens on a Q-tip or finger into the cavity 1504, thereby improving the hygiene of the surgical operation 1500 and reducing the occurrence of injury arising from the application of blunt force. Alternatively, once a small incision is made using scissors, the handpiece (e.g., surgical device 300 of FIG. 3, surgical device 400 of FIG. 4, surgical devices 1100 and/or 1200 of FIGS. 11A, 11B, 12A, and 12B) may be used to cut a slit in the frenulum, avoiding a blunt dissection of the frenumlum.

FIG. 16 shows a surgical operation 1600 involved in a frenectomy or frenoplasty procedure using a surgical device described herein according to one or more embodiments. The surgical operation 1600 involves using the laser light 1410 to dissect or excise posterior parts of the tight frenulum, a genioglossus muscle, fibrous tissue, or fascia 1502 within the frenulum 1404. The surgical devices described herein enable dissection or excision of the genioglossus muscle (or fibrous tissue or fascia) 1502 with reduced risk to bodily tissue around the genioglossus muscle (or fibrous tissue or fascia) 1502, such as blood vessels and nerves. The backstop 1410 can be positioned behind the genioglossus muscle 1502 and the surgical device 1400 can be guided to cut the genioglossus muscle 1502 without damaging surrounding tissue.

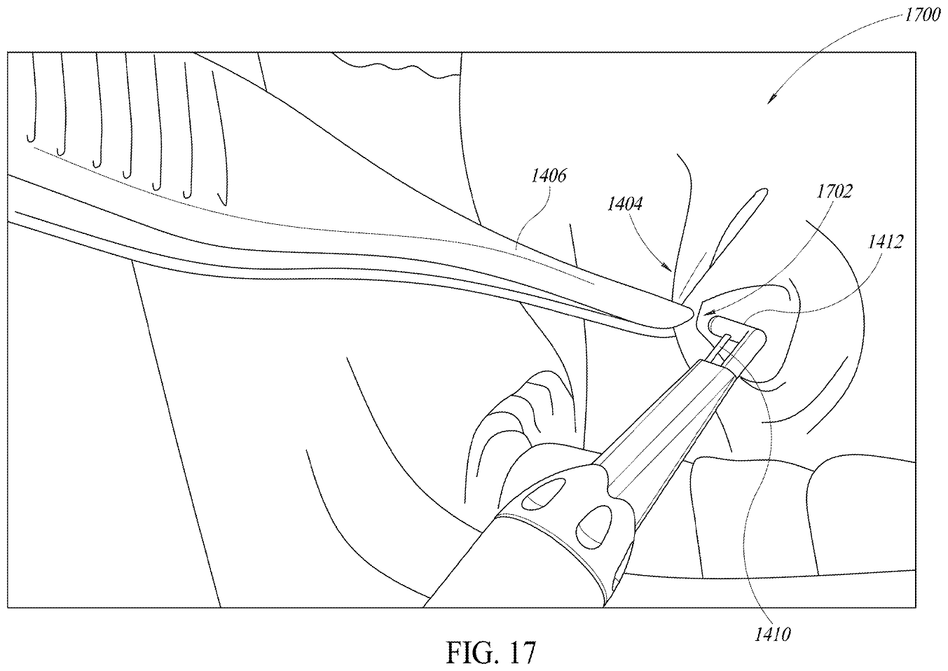

FIG. 17 shows a surgical operation 1700 involved in a frenectomy or frenoplasty procedure using a surgical device described herein according to one or more embodiments. The surgical operation 1700 involves using the forceps 1406 to pull a portion of the frenulum 1404, such as posterior parts of the frenulum, toward the user, and inserting the backstop 1412 behind the frenulum 1404 to cut particular muscle (e.g., fascia, fibers, or muscle) away from the frenulum 1404 and toward a user. The user manipulates the surgical device 1402 such that the laser light 1410 cuts the particular muscle from behind or from the side.

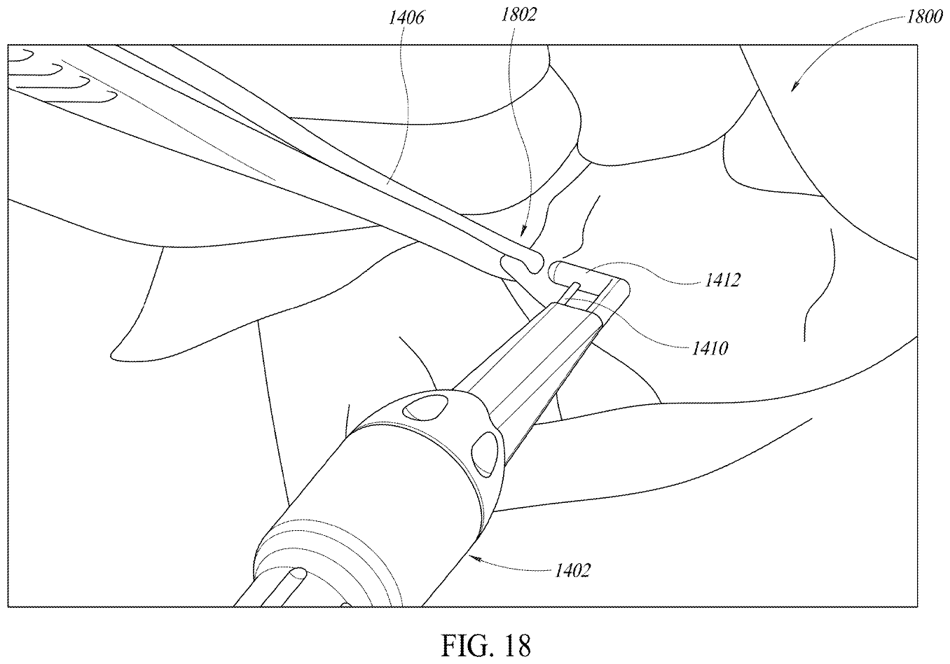

FIG. 18 shows a surgical operation 1800 involved in a frenectomy or frenoplasty procedure using a surgical device described herein according to one or more embodiments. The surgical operation 1800 involves gripping a portion of the frenulum 1404 with the forceps 1406 and excising a portion of the frenulum 1404.

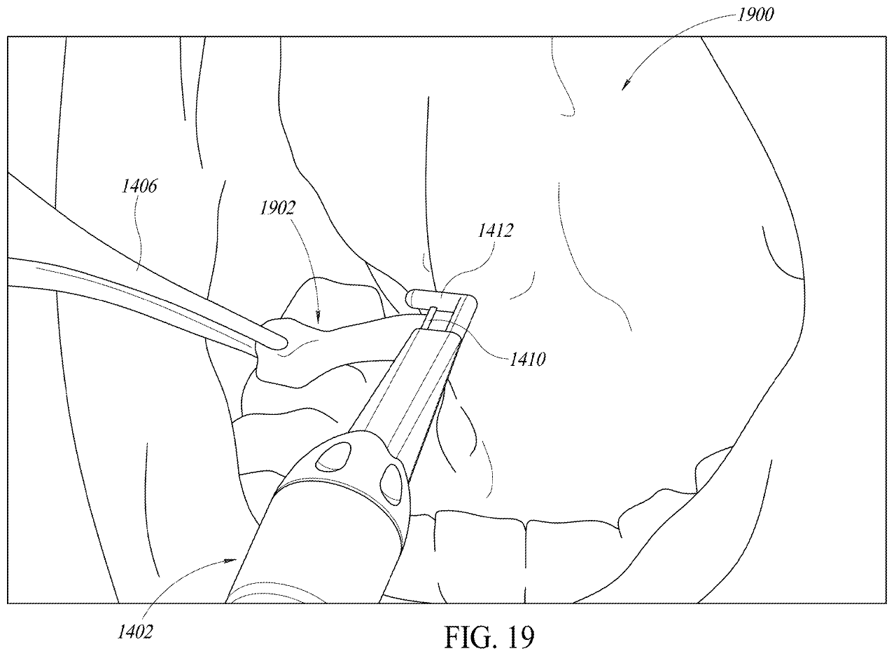

FIG. 19 shows a surgical operation 1900 involved in a frenectomy or frenoplasty procedure using a surgical device described herein according to one or more embodiments. The surgical operation 1900 involved gripping a superfluous portion 1902 of the frenulum 1404, pulling the superfluous portion 1902 taught from the rest of the frenulum 1404, and then cutting away or through the superfluous portion 1902.

In an embodiment, a surgeon may use the handpiece (e.g., surgical device 300, 400, 1100, 1200) to feel inside a patient to identify portions of the frenulum to excise. The patient may be awake during the procedure and the surgeon may manipulate portions of the frenulum using the handpiece and based on the patients response to the manipulation, determine whether to excise that portion of the frenulum. Such methods and devices can be used on a tongue, lips, or buccal frenulae, by way of non-limiting example.

The various embodiments described above can be combined to provide further embodiments. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A surgical device, comprising:
a body having an elongated shape with interior walls defining a cavity extending along a first axis and having a first aperture at a first end of the body, the body configured for attachment of a fiber waveguide;
a backstop spaced apart from the first aperture and having a surface opposing the first aperture along the first axis at a distance of 6 mm or less; and
an arm extending between and connecting backstop and the body, wherein the body includes a second aperture provided at a second end of the body opposite to the first end for receiving the fiber waveguide in the cavity.

2. The surgical device of claim 1, comprising:
a nozzle provided at the first end and having a frustoconical shape, the first aperture located at an end of the nozzle for emitting a laser beam.

3. The surgical device of claim 2, wherein the nozzle is a monolithic structure including at least the nozzle, the arm, and the backstop.

4. The surgical device of claim 1, wherein the backstop extends from the arm and intersects with the first axis, the backstop having a curved surface facing the first aperture.

5. The surgical device of claim 1, wherein the backstop has a body extending upwardly from the arm and an end portion having a hemispherical shape.

6. The surgical device of claim 1, wherein the backstop has a circular cross-sectional area and the surface opposing the first aperture has a flat shape extending in a direction transverse to the first axis.

7. The surgical device of claim 1, wherein the backstop has a circular cross-sectional area and the surface opposing the first aperture has a concave shape extending in a direction transverse to the first axis.

8. The surgical device of claim 1, wherein the backstop and the arm are composed of the same material that forms a monolithic structure.

9. The surgical device of claim 1, comprising:
a wall extending between the first aperture and the arm.

10. The surgical device of claim 9, comprising:
a nozzle portion disposed at the first end and having a shape that tapers in cross-sectional area from the body to the first aperture, wherein the wall extends from the body to the first aperture.

11. The surgical device of claim 9, wherein the arm extends outwardly from the wall at a location spaced apart from the first aperture.

12.-18. (canceled)

19. A surgical device assembly, comprising:
a body having an elongated shape and having first interior sidewalls extending from a first aperture at a first end of the body;
a nozzle attached to the body at the first aperture and including a second aperture, the nozzle portion having second interior sidewalls tapering in cross-sectional area toward the second aperture, the first interior sidewalls and second interior sidewalls defining a cavity extending between the first aperture and the second aperture; and
a backstop portion having a backstop that includes a surface spaced apart from the second aperture at a distance of 6 mm or less and opposing the second aperture.

20. (canceled)

21. The surgical device assembly of claim 19, wherein the nozzle is selectively removable from the body.

22. The surgical device assembly of claim 19, wherein the backstop portion includes an arm extending from the body and connecting the backstop portion to the body.

23. The surgical device assembly of claim 19, further comprising:
an arm that extends from and connects the backstop to the nozzle.

24.-41. (canceled)

42. A surgical device, comprising:
a main body having an elongated shape with first interior walls defining a cavity extending along a first axis and having an aperture at an end of the main body, the cavity sized and shaped to receive a fiber waveguide;
an arm extending from the end;
a backstop spaced apart from the aperture and having a first reflective surface; and
a second reflective surface positioned along the first axis and oriented at an oblique angle with respect to the first axis and oriented at the oblique angle with respect to the backstop.

43. The surgical device of claim 42, wherein the first interior walls define a first portion of the cavity extending along the first axis, the main body includes second interior walls defining a second portion of the cavity extending along a second axis different than the first axis, the first reflective surface spaced apart from and opposing the aperture along the second axis.

44. The surgical device of claim 43, the backstop extending from the arm in a direction transverse to the second axis, wherein the backstop, the arm, and the end define a recessed portion opening transversely relative to the second axis.

45. The surgical device of claim 42, wherein the second reflective surface is provided on the arm and opposes the aperture.

46. The surgical device of claim 45, wherein the backstop opposes the arm, and the first reflective surface opposes the second reflective surface.

47. The surgical device of claim 46, the backstop extending from the end and spaced apart from the arm, wherein the backstop, the arm, and the end define a recessed portion of the surgical device.

48. The surgical device of claim 42, wherein the main body includes a body portion configured to receive the fiber waveguide and includes a nozzle portion having the arm, the backstop, and the second reflective surface, and wherein the nozzle portion is selectively removable and attachable from and to the body portion.

49.-51. (canceled)
